(19) United States
(12) Patent Application Publication
(10) Pub. No.: US 2008/0138865 A1
(43) Pub. Date: Jun. 12, 2008

(54) OPTIMISATION OF CULTURE CONDITIONS FOR PRODUCTION AND PROCESS OF PURIFICATION OF HIGH MOLECULAR WEIGHT HYALURONIC ACID

(75) Inventors: Vidhya RANGASWAMY, Navi Mumbai (IN); Santosh Vyas, Navi Mumbai (IN); Dharmendra Jain, Navi Mumbai (IN); Vedapuri Nataraj, Navi Mumbai (IN); Harshad Velankar, Navi Mumbai (IN); Arnab Kapat, Navi Mumbai (IN); Sonti Venkata Rama Krishna, (US)

Correspondence Address: FOLEY AND LARDNER LLP;SUITE 500, 3000 K STREET NW, WASHINGTON, DC, 20007, US

(73) Assignee: Reliance Life Sciences PVT. LTD.

(21) Appl. No.: 11/774,037

(22) Filed: Jul. 6, 2007

(30) Foreign Application Priority Data
Jul. 6, 2006 (IN) 1065/MUM/2006
Nov. 13, 2006 (IN) 1874/MUM2006

(51) Int. Cl. C12P 19/04

(52) U.S. Cl. 435/101

(57) ABSTRACT

The present invention provides an efficient and improved process for the production and purification of high molecular weight hyaluronic acid (HA). One embodiment relates to providing growth conditions for production of high molecular weight HA in Streptococcus zooepidemicus. For example, growth in medium comprising a lower protein concentration, e.g., 1% casein hydrolyzate, and a higher sugar concentration, e.g., 5% sucrose, gave 5-6 g/L of high molecular weight HA in the range of 3.5-4.0×10⁶ Da. The present invention also provides an efficient process for the purification of HA comprising treatment with silica gel then active carbon, and subsequent diafiltration with less volume of solvent.

### CROSS-REFERENCES TO RELATED APPLICATIONS

This application claims benefit of provisional Indian Application No. 1065/MUM/2006 filed on Jul. 6, 2006 and 1874/MUM/2006, filed on Nov. 13, 2006, which is hereby entirely incorporated by reference.

### FIELD OF THE INVENTION

The present invention relates to improved techniques for producing and purifying hyaluronic acid and its salt. The present invention likewise relates to the production and optimization of process and purification of hyaluronic acid and its salt, having biomedical applications.

### BACKGROUND OF THE INVENTION

Hyaluronic acid (HA) is a naturally occurring biopolymer having biological functions in bacteria and higher animals including humans. Naturally occurring HA may be found in the tissue of higher animals, in particular as intercellular space filler. (Balazs, Viscosupplementation: a new concept in the treatment of osteoarthritis. J. Rheumatol. Suppl.; 39:3-9, (August 1993)). It is found in greatest concentrations in the vitreous humor of the eye and in the synovial fluid of articular joints. (O'Regan, M., Martini, I., Crescenzi, F., De Luca; C., and Lansing, M., Molecular mechanisms and genetics of hyaluronan biosynthesis. International Journal of Biological Macromolecules 16: 283-286 (1994)). In gram-positive Streptococci, it is found as a mucoid capsule surrounding the bacterium.

Hyaluronic acid, also called hyaluronan or hyaluronate, is a glycosaminoglycan distributed widely throughout connective, epithelial, and neural tissues. It is one of the chief components of the extracellular matrix. It contributes significantly to cell proliferation and migration, and may also be involved in the progression of some malignant tumors. Hyaluronan is naturally found in many tissues of the body such as skin, cartilage and the vitreous humor. It is therefore well suited to biomedical applications targeting these tissues. The first hyaluronan biomedical product, Healon®, was developed in the 1970s and 1980s, and was approved for use in eye surgery (i.e., corneal transplantation, cataract surgery, glaucoma surgery and surgery to repair retinal detachment). Other biomedical companies also produce brands of hyaluronan for ophthalmic surgery.

Hyaluronan is also used to treat osteoarthritis of the knee. Such treatments are administered as a course of injections into the knee joint and may act to supplement the viscosity of the joint fluid thereby lubricating the joint, cushioning the joint and producing an analgesic effect. Hyaluronan also may have positive biochemical effects on cartilage cells.

Due to its high biocompatibility and its common presence in the extracellular matrix of tissues, hyaluronan is gaining popularity as a biomaterial scaffold in tissue engineering research. In some cancers, hyaluronan levels correlate well with malignancy and poor prognosis. Hyaluronan may be used as a tumor marker for prostate and breast cancer. It may also be used to monitor the progression of these diseases.

Hyaluronan may also be used postoperatively to induce tissue healing, notably after cataract surgery. Current models of wound healing propose that larger polymers of hyaluronic acid appear in the early stages of healing to physically make room for white blood cells which mediate the immune response. Hyaluronan is a common ingredient in skin care products.

The term “hyaluronate” refers to the conjugate base of hyaluronic acid (HA). Because the molecule typically exists in vivo in its polyanionic form, it is most commonly referred to as “hyaluronan.” HA is comprised of linear, unbranching, polyanionic disaccharide units consisting of glucuronic acid (GlcUA) an N-acetyl glucosamine (GlcNAc) joined alternately by beta 1-3 and beta 1-4 glycosidic bonds, as shown in FIG. 1. HA is a member of the glycosaminoglycan family, which includes chondroitin sulphate, dermatin sulphate and heparan sulphate. Unlike other members of this family, it does not covalently bind to proteins.

In a neutral aqueous solution, due to the hydrogen bond formation, water molecules and adjacent carboxyl and N-acetyl groups impart a conformational stiffness to the polymer, which limits its flexibility. The hydrogen bond formation results in the unique water-binding and retention capacity, of the polymer. It also follows that the water-binding capacity is directly related to the molecular weight of the molecule. It is known that up to six liters of water may be bound per gram of HA. (Sutherland, Novel and established applications of microbial polysaccharides. Trends Biotechnol 16:41-46, 1998).

Hyaluronic acid (HA) and its derivatives have been extensively studied for their applications in biomedical practices. The biocompatibility characteristic of this polymer has attracted attention of orthopedics as it can be used in viscosurgery and allows surgeons to safely create space between tissues. Viscosurgical implants are constructed from HA. (Balazs, August 1993, id.). HA's viscoelastic character has been used to supplement the lubrication in arthritic joints. It has also been used for targeted drug delivery as a microcapsule. Finally, because of its high water retention capacity, this EPS (extracellular polysaccharide) also, occupies a niche in the lucrative cosmetics market. In 2003 the FDA approved hyaluronan injections for filling soft tissue defects such as facial wrinkles. Restylane® is a common trade name for the product. Hyaluronan injections temporarily smooth wrinkles by adding volume under the skin, with effects typically lasting for six months. In addition, HA solutions are characteristically viscoelastic and pseudoplastic. These characteristics are found even in very dilute solutions of this polymer where very viscous gels are formed. The viscoelastic property of HA solutions, which is important to its use as a biomaterial, is controlled by the concentration and molecular weight of the HA. The molecular weight of HA from different sources is highly variable ranging from 10⁴ to 10⁷ Da. The extrusion of HA through the cell membrane as it is produced permits unconstrained polymer elongation, which can result in a very high molecular weight of HA.

Molecular weight is a parameter that most affects the physical properties of hyaluronic acid-based medical devices. Molecular weight influences the viscosity versus concentration profiles of hyaluronic acid solutions, and plays a major role in its other visco-elastic properties. In particular, solutions that require a given viscosity for therapeutic efficacy require lower concentrations of hyaluronic acid if the molecular weight is increased. This has been shown to provide advantages in both ocular and orthopedic applications. A process that is capable of generating medical grade hyaluronic acid having a molecular weight greater than 750,000 daltons thus has significant commercial potential. In many pharmaceutical applications, it is undesirable to have low molecular weight hyaluronic acid in the formulation, for example in view of the inflammatory effects of low molecular weight HA as reported in U.S. Pat. No. 4,141,973

Hyaluronic acid is used in some top selling commercial products, including Synvisc (Genzyme), Orthovisc (Anika), Seprafilm (Genzyme), Restylane (Q-Med), Healon (Pharmacia), Amvisc (Med-Chem), etc. Due to its high cost, however, it is only used in very small concentrations in these products. HA has more commercial value than other microbial EPS. With an estimated world market value of $US 500 million, it is sold for about $US 100 000 per kilogram.

HA has been conventionally extracted from rooster combs and bovine vitreous humor. However, it is difficult to isolate high molecular weight HA at industrially feasible rate from these sources, because it forms complex with proteoglycans present in animal tissue (O'Regan et al., 1994). It is presently impractical to control the molecular weight of the biopolymer while it is synthesized in animal tissue. Moreover, the use of animal-derived biochemicals for human therapeutics has raised ethical issues, and is met with growing resistance. Besides ethical issues, there is a potential infectious disease risk associated with the use of animal-derived biochemicals for human therapeutics. Given that 10 MDa chains could be obtained from animal tissue, there is considerable scope for improvement.

The above discussed drawbacks have forced the industry to adopt to bacterial fermentation processes with the hope of obtaining commercially viable biopolymer. Using a bacterial fermentation process, HA is released into the growth medium, which helps in controlling the polymer characteristics and results in improved HA yields. The amount of biopolymer that can be produced by the above route is theoretically unlimited. The use of bacterial fermentation does have, however, disadvantages. The recent trend is to use Lancefield's group A and C Streptococci, which naturally produce a mucoid capsule of HA. The HA capsule is a biocompatibility factor that enables this gram positive bacteria to evade host immune defenses and accounts for its characteristically high virulence level.

Previous subsequent improvements in the extraction and purification processes using bacteria have resulted in an inherent molecular weight reduction of HA. Streptococcal fermentations have been only able to produce HA with an average molecular weight in the range of 1 to 4 MDa. As discussed above, high molecular weight is a desirable property of the HA bioppolymer. The present invention provides methods for obtaining HA having a high molecular weight.

Streptococci are nutritionally fastidious, facultative anaerobes, which produce lactic acid as a by-product of glucose catabolism. Hence the energy recovered by these bacteria is lower relative to aerobic bacteria. The yield of HA from bacterial fermentation obtained using previously known methods is characteristically low (0.1 g/g glucose, 0.15 g/lh). Such low yields certainly struggle to meet market demand. In addition, strict nutritional requirements for fermentation influence economics by prohibiting the use of chemically defined media for production scale fermentations and limit the choice of complex media that can be employed.

Several groups have provided proposed process parameter optimization, selection of process mode of HA, yield optimizations by complex medium design, and model based fermentation control of HA. Cooney, M. J., Goh, L. T., Lee, P. L., and Johns, R. R., Structure model-based analysis and control of the hyaluronic acid fermentation by Streptococcus zooepidemicus: Physiological implications of glucose and complex-nitrogen-limited growth, Biotechnology Progress, 15: 898-910 (1999); Lars M. Blank, Richard L. McLaughlin, Lars K. Nielsen, Stable production of hyaluronic acid in Streptococcus zooepidemicus chemostats operated at high dilution rate, Biotechnology and Bioengineering, 90(6): 685-693 (2005); Johns, M. R., Goh, L.-T., and Oeggerli, A., Effect of pH, agitation and aeration on hyaluronic acid production by Streptococcus zooepidemicus. Biotechnology Letters 16: 507-512 (1994); Kitchen, J. R., and Cysyk, R. L., Synthesis and release of hyaluronic acid by Swiss 3T3 fibroblasts, Biochemical Journal, 309: 649-656 (1995); Armstrong D, Johns M R, Culture conditions affect the molecular weight properties of hyaluronic acid produced by Streptococcus zooepidemicus, Appl. Environ. Microbiol., 63: 2759-2764 (1997).

Over time, HA molecular weight has become major area of study for researchers. Researchers, such as Armstrong and Johns, have examined the effect of process parameters to ascertain effects on the molecular weight of HA. Armstrong, D. C., and Johns, M. R., Effect of Culture Conditions on Molecular Weight of Hyaluronic Acid Produced by Streptococcus zooepidemicus, Applied and Environmental Microbiology, 63: 2759-2764 (1997); Armstrong, D. C., and Johns, M. R., Improved molecular weight analysis of streptococcal hyaluronic acid by size exclusion chromatography, Biotechnology Techniques, 9: 491-496 (1995).

These groups have found that certain fermentation parameters affect the molecular weight of produced HA. Armstrong et al (1997) reported that conditions such as temperature, aeration and glucose concentration affect the molecular weight of HA whereas pH and agitation do not. It was found that low growth temperatures (28° C.), culture aeration and high initial glucose concentration (40 g/l) resulted in the production of higher molecular weight HA in S. zooepidemicus. Previous groups also found that culture pH and agitation did not affect the molecular weight outcome of HA fermentations. Specifically, Johns et al. (1994) previously reported that pH and agitation affected the production of HA generally, but not molecular weight.

It is already known fact that specific growth rate of the bacteria and the molecular weight of HA are inversely proportional. This effect may be explained by a resource-based metabolic model for HA synthesis, as illustrated in FIG. 2. In its most simplistic form, two competing processes occur within a bacterial cell, i.e., cell growth and biosynthesis of HA. These two processes compete for the limited resources, such as carbon, nitrogen and energy. At low specific growth rates, the cell directs more glucose-derived activated precursors (namely UDP-Glc and UDP-GlcNAc) to HA synthesis rather than cell wall synthesis. The higher ATP yield from aerobic glucose catabolism favors the formation of UTP, which is required for the formation of the two activated precursors of HA synthesis, namely UDP-GlcUA and UDP-GlcNAc. Glucose, which may be used to synthesize HA, is also depleted by lactate production under anaerobic growth.

The specific rate of HA production (g g⁻¹ h⁻¹) was also previously observed to increase with decreasing specific growth rate. Given the fact that the density of active HA-synthesizing enzymes is unlikely to change, the higher rate of production can be attributed to a higher polymerization rate through each synthase. An increased synthase activity may arise from the higher intracellular substrate concentration resulting from low growth rate.

The molecular weight of the HA produced is determined by the number of precursors that the synthase is able to polymerize during its lifetime. Based upon this theory, overexpression of the synthase results in decrease in the molecular weight of HA synthesized. Indeed it may reduce the mean molecular weight because there are more enzymes competing for the same resources. A similar effect was reported for the overexpression of polyhydroxybutyrate synthase in recombinant E. coli. (Sim et al., PHA synthase activity controls the molecular weight and polydispersity of polyhydroxybutyrate in vivo,” Nature Biotech. 15: 63-67, 1997).

U.S. Pat. No. 4,897,349 demonstrated that improved overall yields of HA could be obtained by controlling the oxygen available for metabolism by a hyaluronic acid producing microorganism. The available oxygen was limited to stimulate a disproportionately larger production of the desired hyaluronic acid. The culture conditions with respect to carbon source was not studied. However, the process required modification of dissolved oxygen, which did not usually result in consistent yields of HA produced.

European patent EP 0694616 employed a concentration of 2.5% of a culture of Streptococcus zooepidemicus obtained from a collection. The fermentation took about 24 hours at 37° C., with the pH maintained at 7.00 by continuous automatic addition of NaOH. 33% of the dextrose was added at the moment of inoculation, and the remainder was administered continuously. The yield of the process, as determined by HPLC analysis, was as low as 0.8 g/l with a mean molecular weight estimated at around 500,000 Da.

Japanese patent JP62215397 demonstrated that a microorganism capable of producing hyaluronic acid was aerobically cultured in a medium under agitation, as saccharides were added to the medium several times, the concentration of the saccharides was always kept at 0.1-3 w/v % in the medium. Hyaluronic acid accumulated in the medium was collected. The preferred saccharide was glucose or lactose. The hyaluronic acid accumulated in the culture mixture was separated by a conventional method for separation of polysaccharides. The process presented in this patent was a fed batch process and not applicable to a continuous batch process

Japanese patent JP62257901 disclosed a process for producing HA that could be obtained by culturing hyaluronic acid-producing bacteria in a carbohydrate-containing medium (e.g., with a pH 7.5 containing 0.50% of yeast extract, 0.75% of peptone, 0.02% of sodium sulfite, and 1-3% of glucose).

Japanese patent JP62289198 presented a process wherein a hyaluronic acid-producing microbial strain such as Streptococcus equi NK-850214 was inoculated in a liquid medium containing arginine and/or glutamic acid at a concentration of 0.01-1 wt %, in addition to peptone, NaCl, trace vitamins, etc., by a culture process comprising the addition of ≧20 g carbohydrate, such as glucose per 1 liter of the medium, in ≧3 divided doses in a manner that constantly kept the carbohydrate concentration at 0.1-3 w/v % throughout the culture.

In Japanese patent JP05276972, a microorganism capable of producing hyaluronic acid was cultured in a culture solution containing a surfactant. Hyaluronic acid was formed and accumulated in the culture solution and collected.

Japanese patent JP06038783 provided a process of producing hyaluronic acid on an industrial scale by culturing a hyaluronic acid-producing microbial strain belonging to the genus Streptococcus in a nutrient medium. The medium contained a specific amount of a sugar component (as the main carbon source) under specific conditions while stirring the medium by aeration. A microbial strain belonging to the genus Streptococcus and capable of producing hyaluronic acid (e.g., Streptococcus zooepidemicus) was inoculated on a nutrient medium containing ≧3% sugar component as a main carbon source and was cultured at pH 7 and 33° C. for 4 days under stirring by aeration. The yield obtained by this process was 2.1 g/L.

Japanese patent JP06319579 presented the production of hyaluronic acid in a medium containing glucose and fructose as major carbon source to accumulate hyaluronic acid of high molecular weight in the culture mixture.

Japanese patent JP2000189185 provided a method of producing hyaluronic acid in high yield, stable in an industrial scale, by culturing a microorganism capable of producing hyaluronic acid. The culturing operation was continued until the concentration of HCO₃⁻ ion was maintained at 5-500 ppm in the culture mixture.

U.S. Pat. No. 4,784,990 demonstrated a method for obtaining sodium hyaluronate comprising growing with vigorous agitation a microorganism of the genus Streptococcus under appropriate conditions in a nutrient medium containing a sugar component as a carbon source. The '490 patent concerned a method for selecting microorganisms which produce enhanced amounts of hyaluronic acid and which lack hemolytic activity, such as S. zooepidemicus HA-116 ATCC 39920 which was a mutant strain. The S. zooepidemicus HA-116 ATCC 39920 microorganism produced large amounts of high molecular weight hyaluronic acid. The sodium hyaluronate was then recovered by removing the microorganism and other materials insoluble in the medium, precipitating the sodium hyaluronate from the medium, e.g. using organic solvents, and recovering the precipitate. The precipitate was then ground and dried. Compositions of sodium hyaluronate characterized by an absence of pyrogenicity and inflammatory activity could be produced by these methods. The medium had a substantially constant pH between about 6.0 and 7.5 and the sodium hyaluronate excreted into the medium by the organism was purified using methods involving precipitation, redissolving and reprecipitating the hyaluronate. The sodium hyaluronate could be precipitated from the medium or filtrate by adding a first organic solvent, such as isopropanol, to the medium. The precipitate was redissolved in 3% aqueous sodium acetate and then reprecipitated with a second organic solvent such as ethanol. The second precipitate was redissolved in 3% aqueous sodium acetate and activated charcoal was added to form a suspension. The suspension was filtered and a third organic solvent, e.g. acetone, was added to produce a precipitate of sodium hyaluronate. The first, second and third organic solvents could each be isopropanol, ethanol or acetone. Alternatively the hyaluronate could be precipitated by the same organic solvent in each step. For example, sodium hyaluronate was precipitated from the medium by using isopropanol in all three of the precipitation steps. The process in this patent for medical grade HA also involved the use of detergents such as cetyl pyridinium chloride, multiple solvent precipitation steps, as well as florisil treatment.

U.S. Pat. No. 4,946,780 provided a method of producing HA from fermentation by employing surfactants such as cetyl pyridinium chloride. The method also involved a purification procedure comprising contacting the solution containing sodium hyaluronate with alumina, activated charcoal or mixtures thereof and then with silica to form a purified solution of sodium hyaluronate. The method then added alcohol to the purified solution of sodium hyaluronate to precipitate purified sodium hyaluronate and dried the purified sodium hyaluronate.

U.S. Pat. No. 5,563,051 disclosed a process wherein after the fermentation process, the biomass was killed with a particularly suitable agent. The agent used to kill the biomass, was formaldehyde, e.g., in the form of the aqueous solution commonly known as formalin. The HA was extracted with an aqueous medium containing an anionic surfactant, i.e., sodium dodecyl sulphate. The patent further disclosed the use of an ultra filtration membrane with an appropriate molecular weight cut off which was usually from 10,000 to 25,000 Daltons, and preferably 20,000 Daltons. The filtered solution containing the dissolved HA was diafiltered with 8 to 20 volumes of purified water, preferably about 10 volumes of purified water, where the filtrate was continuously discarded.

U.S. Pat. No. 6,489,467 claimed a process for purifying high molecular weight hyaluronic acid from a biological source, including the steps of adjusting the pH of an aqueous solution containing high molecular weight hyaluronic acid from a biological source to a pH in the range from 1.7 to 3.3. The process further involved diafiltering the aqueous solution at the same pH using a filter having a pore size in the range from 100,000 Daltons nominal molecular cut-off to 0.45μ, and removing cells from the aqueous solution containing high molecular weight hyaluronic acid from biological source.

U.S. Pat. No. 7,002,007 disclosed methods involving contacting a hyaluronate-containing source with an acid to make an acidic hyaluronate suspension, contacting that suspension with an anionic exchange medium in the presence of an acidic buffer, and thereafter contacting the medium with an acidic buffer having a higher salt content to desorb the hyaluronate from the medium.

Previous methods for producing HA usually use glucose as a carbon source. Using these methods, the molecular weight of the HA obtained often was found to be between 2 and 4×10⁵ Da, i.e., a relatively low molecular weight. Moreover, the inherent viscosity of HA increases with the increase in its molecular weight. This increased viscocity has presented a major obstacle during fermentation, as well as in purification of HA. Although high molecular weight HA with may be too large to penetrate the skin and the bloodstream, it has a number of valuable uses. In in topical preparations, for example, high molecular weight HA is useful in eye surgery and in cosmetics. Likewise, high molecular weight HA can be used in a viscoelastic film to retain skin moisture and to block foreign substances.

Therefore, it has become a challenge and a necessity to provide a process for producing high molecular weight HA, including that which meets specifications set for medical applications, e.g., as described in British Pharmaacoepia, 2003.

Previous methods for purifying HA have involved at least three successive solvent precipitation steps, thereby resulting in an increased number of process steps and an overload on the lyophizer. Previous methods for precipitating fermentation broth also involved the use of surfactants or detergents, such as cetyl pyridinium chloride/hexadecyltrimethyl ammonium bromide. The removal of residual detergent or surfactant increases number of processing steps needed, it has posed complexity and cost.

In addition, previously known purification procedures, such as diafiltration steps employed to achieve the high molecular weight HA involve the use of large volumes of organic solvent. The use of such solvent creates handling problems in scale up operations and results in the environmental hazards.

The present invention relates to improvements on methods for producing and purifying HA, such as high molecular weight HA and its salt, including that having medical applications as in British Pharmaacoepia, 2003. The present invention addresses a simple and economically feasible process for production high yield of HA by continuous fermentation of bacteria, such as Streptococcus zooepidemicus, in a suitable culture medium to produce high yields of high molecular of HA. The process of the present invention is advantageous over traditional batch culture in which degradation enzymes can begin to break down the cell walls of Streptococcus, and release cell contents into the fomenter broth, leading to purification difficulties of HA. Furthermore, the present invention also presents a purification process of hyaluronic acid that includes silica gel filtration combined with active carbon treatment and subsequent diafiltration using less amount of solvent, thereby yielding a better quality of hyaluronic acid having biomedical applications. Likewise, the present invention provides a commercially viable purification process of hyaluronic acid and its salts thereof, having industrial applications.

### OBJECT OF THE INVENTION

It is the object of the present invention to provide a process of producing hyaluronic acid meeting the specifications set for medical applications in Pharmacoepia.

Likewise, it is the object of the present invention to provide a commercially viable process for producing hyaluronic acid and its salt.

It is the object of the present invention to provide a high molecular weight hyaluronic acid of medical grade produced by S. zooepidemicus.

It is the object of the present invention to provide a high molecular weight hyaluronic acid without the use of surfactants or detergents in the manufacturing process of HA.

It is the object of the present invention to optimize the effect of various conditions such as carbon source, metal ion and media composition on the molecular weight of HA, under the present invention.

It is the object of the present invention to provide purification process of hyaluronic acid, yielding a better quality of hyaluronic acid having biomedical applications.

It is the object of the present invention to provide an efficient process for removal of protein impurities from hyaluronic acid by employing two steps of silica gel purification and carbon treatment.

It is the object of the present invention to provide an efficient process for diafiltration using less amount of solvent in the purification of hyaluronic acid.

It is the object of the present invention to provide a high molecular weight hyaluronic acid of medical grade.

It is the object of the present invention to provide a process that is free from surfactants or detergents.

It is the object of the present invention to provide a single cycle process for purification of hyaluronic acid.

It is the object of the present invention to provide a process, which comprises minimal amount of solvents.

It is the object of the present invention to remove at least 90% of the protein impurities employing silica gel and activated carbon treatment.

It is the object of the present invention to provide a process for constant volume diafiltration resulting in high molecular weight HA.

It is also the object of the present invention to provide HA as per specifications of British Pharmacoepia 2003.

### SUMMARY OF THE INVENTION

The present invention provides a cost effective process of producing high molecular weight hyaluronic acid and its salt. The present invention yields a better quality of hyaluronic acid having biomedical applications, free from surfactants or detergents.

The present invention comprises the following steps for the production of medical grade hyaluronic acid.
a) Preparation of HA by fermentation
b) Removal of protein impurities
c) Diafiltration

In one embodiment, the present invention uses fermentation technique for the production of high molecular weight hyaluronic acid using bacteria. In one embodiment, the bacterium is Streptococcus zooepidemicus.

In another embodiment, the present invention uses a carbon source, metal ion and media composition that optimize the production of high molecular weight HA.

In one embodiment, HA is prepared by initial culturing of the microorganism Streptococcus zooepidemicus, in a suitable medium followed by fermentation of the broth in a 1-10 Liter fermentor at about 30-40° C., with agitation at about 300-500 rpm and 1-3 vvm (vol/vol/min) aeration for about 15-28 hours by maintaining the pH at a neutral range. (Aeration rate vvm=gas volume flow per unit of liquid volume per minute (volume per volume per minute)). After incubation, the fermentation broth is suitably diluted with water and clarified. The HA present in the clarified broth is reprecipitated with equal volumes of suitable solvent including but not limited to isopropanol. The precipitated high molecular weight HA is then converted to its salt wherein it can be then solubilised for subsequent purification procedure. In one embodiment, the HA is converted into its sodium salt by the addition of 3% sodium acetate and homogenized until complete dissolution.

In one embodiment, the present invention provides culture conditions that optimize the yield and molecular weight of HA. It has been found that production of HA is a growth associated phenomenon. One can produce HA with high molecular weight by controlling culture conditions. For example, the molecular weight of produced HA increases with bacteria growth. At the beginning of fermentation process, lower molecular weight population (˜5000 Da) is about 60% of total HA. About 70% of total HA is of high molecular weight (>800 KDa) by the end of 22 h of fermentation (FIG. 4).

In addition, one may use different metals in the fermentation process of HA. Metal ions are known to enhance capsule production in various bacteria. The effect of CuSO₄, MnSO₄ and ZnSO₄ at 0.025% (final conc.) was tested on the yield of HA at the end of 24 h of fermentation. While there was no significant difference between the control (no metal addition) and the flasks treated with CuSO₄ or MnSO₄, a significant portion (60%) of total HA in the medium treated with ZnSO₄ was found to be in the higher molecular weight region (>800 KDa) (FIG. 5).

Likewise, the carbon source, i.e., sugar, in, the culture medium may be varied. The growth and HA production in medium containing various types of sugars were tested. Different sugars in culture medium affected the molecular weight of HA during production. While growth was fairly constant in most of the sugars (2% final conc.), lactose and sucrose gave better yield of high molecular weight HA (>800 K) as compared to glucose, which gave HA of lower molecular weight (FIG. 6). The yield of HA obtained using sucrose was at least 10-fold higher and molecular weight of HA was found to be much higher, as compared to glucose. As shown in FIG. 6, the molecular weight of HA using sucrose by the present invention was greater than 8×10⁵ Da (i.e., 800 kDa), while in glucose medium, the molecular weight was found to be between 2 and 4×10⁵ Da (i.e., 200-400 kDa).

The effect of media on the production of high molecular HA was examined. In one experiment, sugar and casein hydrolyzate enzyme content in the medium were varied. In this experiment, sugar provided necessary energy and casein enzyme hydrolysate provided a necessary nitrogen source. FIG. 7 shows results when the sugar concentration was increased from 2% to 5% and when casein hydrolyzate enzyme concentration was brought down to 1% from 2.5% in the medium. The parameters provided an increased yield of 5-6 g/L of HA (FIG. 7). “Sugar” in FIG. 7 refers to sucrose; “biomass” refers to the cell density, which reflects growth of the microorganism. The yield was less when both the sugar concentration was lower (2%) and casein enzyme hydrolysate concentration was higher (2.5%), or when only the sugar concentration was increased to 5% (data not shown).

In one embodiment, HA is prepared by initial culturing of the microorganism Streptococcus zooepidemicus, in a suitable medium and subsequent fermentation of the broth in a 1-10 Litre fermentor at about 30-40° C., with agitation at about 300-500 rpm and 1-3 vvm aeration for about 15-28 hours by maintaining the pH at a neutral range.

In one embodiment, the present invention provides an efficient process for removal of protein impurities from hyaluronic acid. The present invention employs silica gel filtration and active carbon treatment for efficient removal of protein impurities.

In one embodiment the present invention provides an efficient process for purification of Hyaluronic acid by diafiltration. The present invention employs less amount of solvent in the process.

After incubation, the fermentation broth is suitably diluted with water and clarified. The HA present in the clarified broth is reprecipitated with equal volumes of suitable solvent including but not limited to isopropanol.

The precipitated high molecular weight HA is then converted to its salt wherein it can be then solubilized for subsequent purification procedure. The present invention converts the HA into its sodium salt by the addition of 3% sodium acetate and homogenized until complete dissolution.

The present invention also provides an efficient purification process that removes protein impurities from hyaluronic acid. The process employs silica gel filtration and active carbon treatment for efficient removal of protein impurities. The initial treatment with silica gel removes about 68-70% protein. After removal of silica gel by centrifugation, the high molecular weight HA is treated with active carbon, which then removes 85-90% of the protein. In one embodiment, the process involves passing the silica treated sample through a carbon impregnated on cellulose cartridge.

In one embodiment, the solution obtained after removal of protein impurities is further purified by diafiltration. The present invention employs less amount of solvent in the process. For example, in one embodiment, the diafiltration step involves dilution of the HA solution with the solvent, i.e. pyrogen-free water, and the dilution is done only 5 times, as compared to 10 times, as described in U.S. Pat. No. 6,489,467 (Carlino et al. (2002), Process for purifying high molecular weight hyaluronic acid) thereby significantly reducing the volume handling. A continuous mode of diafiltration may be used, which involves dilution with sterile, pyrogen free water, for example five times. In one embodiment, the process further involves isolate sterile, purified hyaluronic acid by filtering through a 0.22 μm pore filter. In another embodiment, sodium hyaluronate product for biomedical purposes can be obtained by aseptic filtration.

Optionally, the sodium hyaluronate can be reprecipitated with isopropanol to yield the purified high molecular weight HA. The HA obtained can be lyophilized until the moisture content is less than 5%.

In sum, the present invention provides an improved purification process for HA that does not employ acidic pH conditions, and does not use any detergents nor surfactants. The removal of protein impurities step does not employ any hazardous chemical, such as formalin. In addition, the purification process employs less solvent dilutions and therefore reduces the load on the lyophilizer.

### BRIEF DESCRIPTION OF THE DRAWINGS

The following drawings form part of the present specification and are included to further demonstrate certain aspects of the present disclosure. Thus, the invention can be better understood by reference to one or more of these drawings in combination with the detailed description of embodiments presented herein.

FIG. 1: Shows disaccharide repeating unit of HA comprising GlcUA and GlcNAc.

FIG. 2: Shows the HA biosynthetic pathway in Streptococci.

FIG. 3: Illustrates the flow sheet of the process for preparation of high molecular weight hyaluronic acid of medical grade.

FIG. 4. Distribution of HA of various molecular weight during the growth of S. zooepidemicus.

FIG. 5. Effect of metal ions on population of HA of varying molecular weights.

FIG. 6. HPLC profile of HA from media containing different carbon sources. Culture medium was supplemented with glucose, sucrose, lactose or fructose at a final concentration of 2%.

FIG. 7. Medium optimization for increased yield of HA. Initial sucrose content was 5%, and the concentration of casein hydrolyzate was 1.0%. The “%” corresponds to g/100 ml of medium; “sugar” refers to sucrose; and “biomass” is the cell density reflecting growth of the microorganism. Casein hydrolyzate enzyme acts as a nitrogen source during growth of the bacteria.

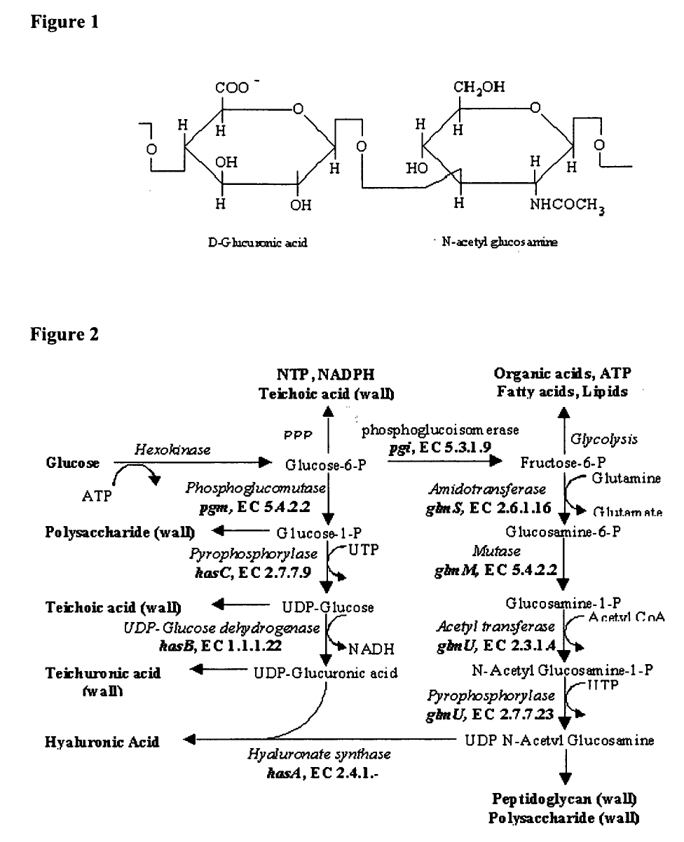

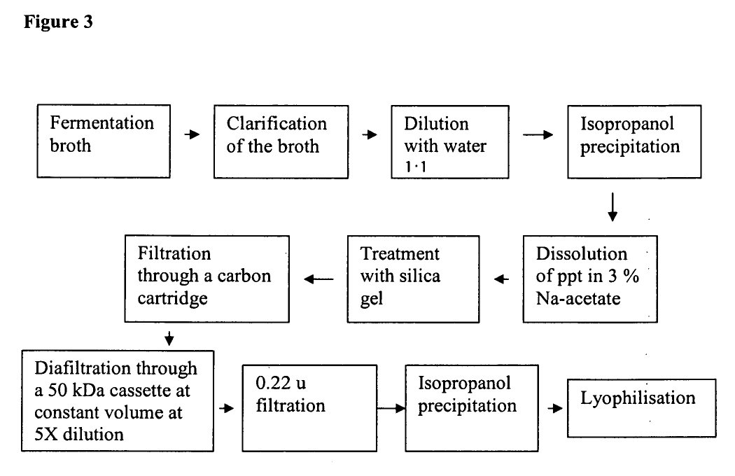

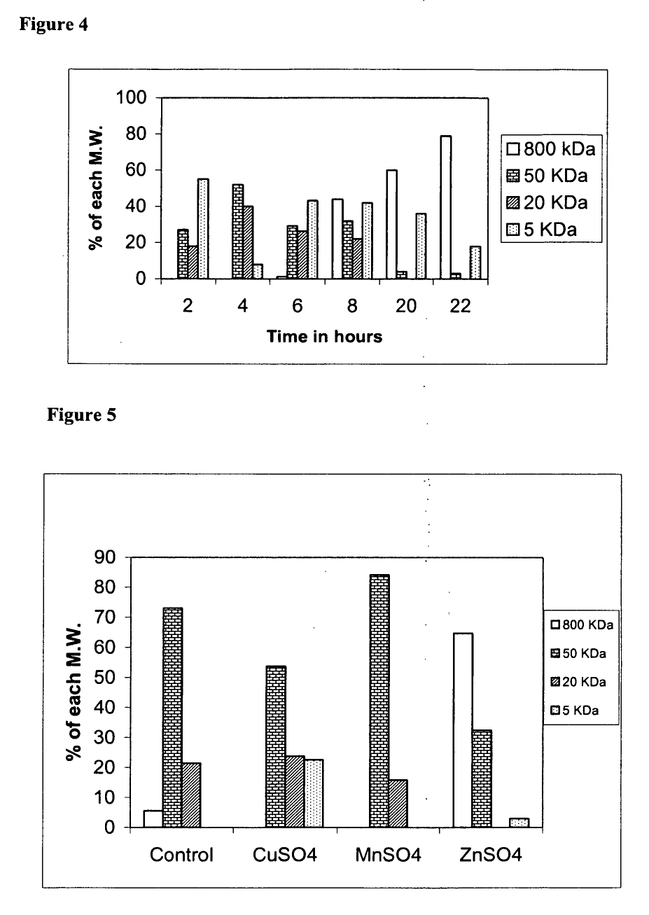

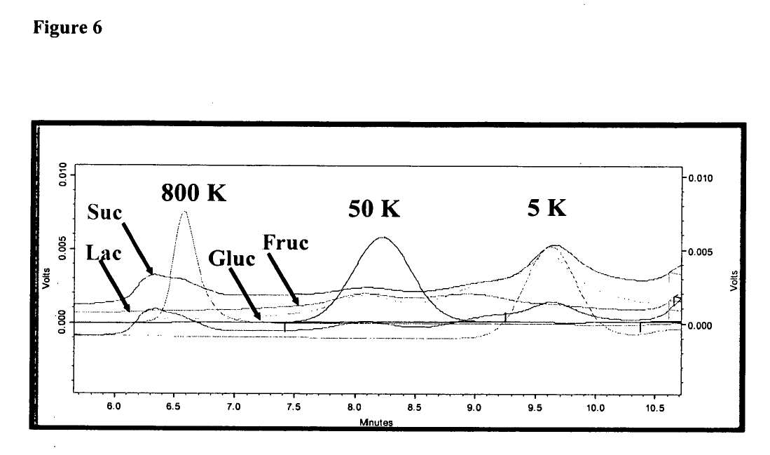

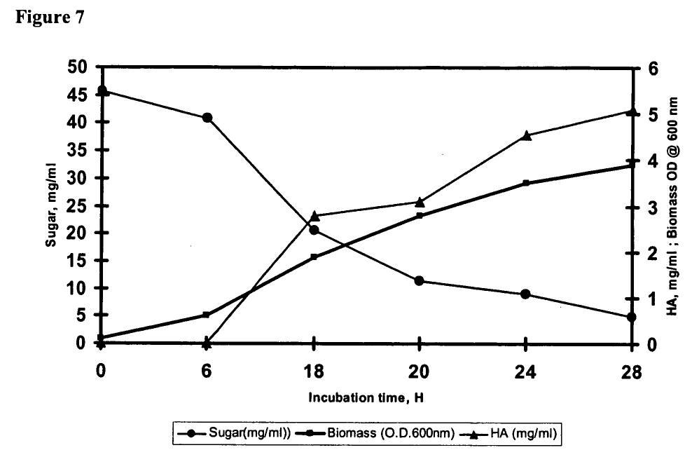

### DETAILED DESCRIPTION OF THE INVENTION

The present invention provides methods for obtaining high yield of medical grade high molecular weight HA from bacterial fermentation broth. The present invention also provides a cost effective methods for purifying HA.

### Definitions

The term “hyaluronic acid” or “HA” as used herein indicates hyaluronic acid obtained from any biological source.

The term “hyaluronate” as used herein refers to any salt form of HA, such as the sodium salt of HA.

The term “high molecular weight” as used herein refers to HA or a salt of HA having a molecular weight of at least 1000 KDa. (1 million Da)

The term “silica gel” refers to a porous, granular form of silica, synthetically manufactured from sodium silicate with varying porosities. One example is a silica gel sold by Ineos.

“Active carbon” (also known as activated charcoal) comprises carbon material, often derived from charcoal. It refers to a material that has a high surface area and exhibits high microporosity. One example is sold by Millipore (Millistak +, Activated Carbon minicapsule M40AC23HH3).

The term “diafiltrating” or “difiltration” refers to a process that may be used for purifying large molecular weight species, increasing the recovery of low molecular weight species, buffer exchange or simply changing the properties of a given solution. A crossflow filtration process allows for the transfer of low molecular weight species, water and/or solvents through a membrane without changing the solution volume. Difiltration can be performed, for example, by Sartocon slice cassette mod. No. 3051465001E-SG, 50 kDa MWCO.

The term “neutral pH” means refers to a pH of about 7.

The term “solvent” used herein indicates any solvent organic or inorganic solvents. In one embodiment, the solvent is isopropanol.

In one embodiment, the microbial source is a Streptococcus species capable of producing high molecular weight hyaluronic acid. For example, the microbial source may be selected from Streptococcus zooepidemicus, Streptococcus equi and Streptococcus pyogenes.

As noted previously, production of HA is a growth associated phenomenon and is inversely related to the growth. In one embodiment of the present invention, culturing and fermentation parameters are selected that balance these two processes.

The molecular weight of HA increases over time in culture, with smaller chains being detected early in the growth phase followed by high molecular weight HA being detected during the latter part of growth. Previous studies, such as those by Armstrong and Johns, have suggested that temperature, pH and agitation speed affect molecular weight of HA drastically when producing HA in bacteria. When comparing results with that of the study carried out by Armstrong et al. (Armstrong and Johns, 1997) and other conventional processes, other researchers found that the culture pH and agitation speed did not affect the molecular weight of HA drastically during production.

In the present invention, the use of lower temperature and aeration, amongst other parameters, can improve the yield of HA production, and increase the molecular weight of HA that is produced.

Previous studies found that initial glucose concentration had a profound effect on the molecular weight of HA. One group, Armstrong and Johns, offered an explanation, i.e., that when the activated sugar monomers UDP-GlcUA and UDP-GlcNAc were present at high concentrations, HA chain elongation persisted. The external glucose concentration might have been linked to the internal concentrations of these monomers, because they were derived from glucose and their synthesis requires substantial energy consumption. (Armstrong and Johns, 1997).

In the present invention, however, inventors have found that sucrose and lactose are better carbon sources than glucose for production of high molecular weight HA. Because sucrose and lactose are disaccharides, they may serve as better energy source than glucose. The present invention in one embodiment further provides that growth on sucrose or lactose at a higher concentration, and protein, such as casein hydrolyzate, at a lower concentration, generates 5-6 g/L of high molecular weight HA. The molecular weight of HA was found to be in the range of 3.5−4.0×10⁶ Da.

The effect of various metal ions on the molecular weight of HA produced have also been studied. The metal ions present in the culture medium are responsible for oxidative stress to the bacteria whereby free radicals are being released during the process, which can have a bactericidal effect. In order to circumvent, several bacteria, particularly some Pseudomonas species produce EPSs (extracellular polysaccharides) such as alginates, etc. which helps the bacteria top invade the host and thus survives. Keith L M W, Bender C L. AlgT (ζ²²) Controls Alginate Production and Tolerance to Environmental Stress in Pseudomonas syringae, J. Bacteriol., 181: 71767184 (1999).

At least one group has suggested that streptococcal and mammalian hyaluronate synthases prefer magnesium ion to other metal ions for the production of high molecular weight of HA. (Yamada and Kawasaki, Microbial synthesis of hyaluronan and chitin: new approaches. J. Biosci. Bioeng. 99: 521-528, 2005). Yamada T and Kawasaki, 2005 provided another novel HA producing Chlorella-virus system stimulated by manganese ions, which produced about 0.5-1 g/L of HA. In the present invention, metal ions such as Cu and Mn do not induce the production of high molecular weight HA, although significant population of HA in the medium containing Zn were in the high-molecular weight range.

To summarize, the present invention has optimized the culture conditions that have resulted in improved yield, such as 5-6 g HA/L, of high molecular weight HA. In one embodiment, casein hydrolyzate enzyme is lowered and the sugar concentration is increased in the medium. These conditions result in lower growth rate. Because production of HA is inversely proportional to growth, increase in HA yield is obtained. In one embodiment, the molecular weight of HA obtained by the present invention is about 3.5×10⁶ to 3.9×10⁶ Da after 24 h of fermentation.

In one embodiment of the present invention, a process for producing high molecular weight hyaluronic acid (HA) or its salt having a molecular weight of at least 1 million Da, where the process comprises: (a) providing a bacteria capable of producing HA; (b) providing a nutrient medium comprising a: divalent metal ion salt (such as MgSO₄, CuSO₄, MnSO₄, and ZnSO₄), casein hydrolyzate, and a carbon source (such as lactose or sucrose); (c) culturing the bacteria in the nutrient medium; and (d) fermenting, such as continuous fermenting, the nutrient medium under aerobic conditions. Bacteria capable of producing HA include those selected from the group consisting of Streptococcus zooepidemicus, Streptococcus equi and Streptococcus pyogenes.

In another embodiment, the nutrient medium comprises 1% casein hydrolyzate and/or 5% of lactose or sucrose. In another embodiment, the nutrient medium comprises zinc, such as in the form of ZnSO₄, casein hydrolyzate at a concentration of 10 g/L, and a carbon source selected from the group consisting of lactose and sucrose at a concentration of 50 g/L. In another embodiment, the fermenting step is conducted for at least 15 hours at a temperature of 30-40° C., an agitation speed of 300-500 rpm, an aeration of 1-3 vvm, and at neutral pH.

In another embodiment, the process produces HA or its salt having a molecular weight in the range of about 3.5×10⁶ Da to 4.0×10⁶ Da. In one embodiment, the process yields at least 5 g of high molecular weight HA per liter of nutrient medium. In one embodiment, the molecular weight of the HA is at least 3×10⁶ Da after 24 hours of fermenting.

The present invention also provides a method for the removal of protein impurities from produced HA, which comprise the two steps of silica gel treatment followed by active carbon treatment. A subsequent diafiltration step also helps ensure a highly purified salt of HA. The product thus obtained can be then aseptically filtered for biomedical applications.

Previously known purifying processes precipitate HA using detergents or surfactants, such as cetyl pyridinium chloride/hexadecyltrimethyl ammonium bromide. This requires, however, multiple steps to remove the residual detergent or surfactant. The present invention provides an efficient purification method for HA, which does not employ detergents or surfactants.

Previously known processes for purifying HA using diafiltration techniques have involved a high number of dilutions with solvent, such as diluting 10 times. See U.S. Pat. No. 6,489,467 (Carlino, et al., Process for purifying high molecular weight hyaluronic acid (2002)). The use of high volumes of solvent creates problems during handling at a larger scale, such as bigger processing vessels and longer time to complete the process and of course higher cost of production. The present invention avoids such problems by using fewer dilutions.

In one embodiment, a process for purifying hyaluronic acid (HA) or its salt from a bacterial fermentation broth comprises: (a) diluting and clarifying the fermentation broth; (b) precipitating the HA present in the broth with a solvent, such as isopropanol, ethanol or acetone, in amount equal to or less than the volume of the diluted and clarified broth of step (a); (c) dissolving the precipitated HA or its salt in a solution, such as 3% sodium acetate; (d) adding silica gel to the HA solution or HA salt solution from step (c) and then removing the silica gel, such as by centrifugation; (e) treating the HA solution or HA salt solution from step (d) with active carbon; and (f) diafiltrating the HA solution or HA salt solution from step (e) using a solvent, such as sterile pyrogen-free water, in an amount no more that five times the total volume of the solution from step (e); wherein the process is conducted in the absence of any detergent or surfactant or formalin, and at neutral pH.

In another embodiment, the process further comprises converting the HA precipitated in step (b) to its salt, and then homogenizing the HA salt in the solution of step (c). In another embodiment, the active carbon is impregnated on cellulose cartridge in step (e). In another embodiment, the process further comprises isolating sterile, purified HA or its salt by filtering through an aseptic filter; and/or lyophilizing the HA or its salt until the moisture content is less than 5%.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Bacterial Strain and Media

Streptococcus equi subsp. zooepidemicus ATCC 39920 was obtained from American Type Culture Collection. The bacteria were maintained on brain heart infusion agar or tryptic soy broth.

### EXAMPLE 2

### Estimation of HA

HA was routinely estimated by the carbazole assay (Bitter, T., and Muir, M., A modified uronic acid carbazole reaction. Anal Biochem 4: 330-334, 1962) in the fermented broth after precipitation with equal volume of isopropanol and redissolving in 3% Na-acetate solution.

### EXAMPLE 3

### Media Optimization

For media optimization experiments, the S. zooepidemicus was grown in a media constituting 2.5% casein hydrolyzate enzyme, 1% yeast extract, 0.2% K₂HPO₄, 0.15% NaCl, 0.04% MgSO₄.7H₂O, and 2% carbon source (sucrose). The organism was grown in Erlenmeyer flasks at 37° C. at 200 rpm for 24 h. For evaluating the effect of metal ions on HA production, filter sterilized solutions of CuSO₄, ZnSO₄ and MnSO₄ were added to the medium at final concentration of 0.025%. See FIG. 5.

### EXAMPLE 4

### Molecular Weight Determination

The molecular weight of HA was determined by size-exclusion chromatography on HPLC using a Shodex OH-Pak SB805-804HQ columns connected in series. The mobile phase used was 1 M NaNO₃ at 1 ml/min flow rate and peaks were detected using a R1 detector. The column was calibrated with pullulan standards of varying molecular weights.

### EXAMPLE 5

### Removal of Protein Impurities

### Silica Gel Treatment

A portion of the homogenized suspension, 100 ml, was treated in batch mode, with silica gel at 2% final concentration for adsorption of protein. This step removes 68-70% protein. The silica gel is separated from the sodium hyaluronate solution by centrifugation at 12,000 rpm for 20 min at 4° C.

### Active Carbon Treatment

Further treatment of high molecular weight hyaluronic acid solution was done with charcoal adsorbed 0.45μ filter assembly (Millipore, Millistak +, Activated Carbon minicapsule M40AC23HH3). The flow rate was 14 ml/min. This step removed 85-90% of the remaining protein.

### EXAMPLE 6

### Purification

### Continuous Mode Diafiltration

The flow through from carbon filter was further purified by using continuous mode diafiltration process. Diluted HA solution was pumped (Flow rate 15 ml-20 ml/min) in to cross flow filter holder equipped with 50 kDa cut off polyether sulphone cassette. (Sartocon slice cassette mod. No. 3051465001E-SG). At the beginning of the feeding of the aqueous broth in to filter, the permeate valve was closed in order to recirculate the aqueous broth for several times until the system was stable and no bubbles seen in retentate. The permeate valve was opened after 10 min and the aqueous broth was recirculated in the feed reservoir for additional 10 min to ensure no leakage of HA occurs across the membrane through the permeate valve. After 15 min. permeate and retentate were collected separately. Five equivalent volumes of sterile pyrogen free distilled water were added continuously in the reservoir containing HA solution. This addition of water to HA solution was done at same flow rate as the outflow rate of the filtrate. The inlet pressure of the cross flow filter holder was maintained around 0.5-1.5 bar. The retentate was used for further recovery of hyaluronic acid and permeate was discarded. Essentially, the permeate contained no HA. The retentate was concentrated to original volume and analyzed for purity. The diafiltration step removes 80-85% of the remaining protein.

### Aseptic Filtration

The concentrated hyaluronic acid solution from diafiltration process is finally subjected to 0.22 μm filtration aseptically. (Millipore stericup: SCGPU02RE, PVDF filter material) which renders the product that qualifies for biomedical application. The product thus formed was lyophilized to obtain medical grade high molecular weight sodium hyaluronate.

### EXAMPLE 7

### Optimized Parameters of Preparation and Purification of HA

Streptococcus zooepidemicus ATCC 39920 was cultured in a medium composed of 1% yeast extract, 1% casein hydrolyzate enzyme, 0.2% K₂HPO₄, 0.15% NaCl, 0.04% MgSO₄.7H₂O, and 5% sucrose. The organism was grown in 1 L medium at 37° C. at 400 rpm for 24 h at 1 vvm aeration. The pH of the medium was maintained at 7.0 by continuous addition of NaOH aqueous solution. After the incubation, the broth was diluted with 1 volume of water and clarified by centrifugation at 10,000 rpm for 20 min at 4° C. The HA in the clarified broth was precipitated with equal volume of isopropanol. The precipitate separated by centrifugation at 10,000 rpm for 20 min at 4° C., is suspended in 1 L of 3% sodium acetate using a mechanical homogenizer (Kinematica-A.G., polytron P T 2100) at 15000 rpm for 10 min×3 cycles. The homogenized solution is treated with silica gel at 2% final concentration for adsorption of protein. This step removes 85% protein. The silica gel is separated from the sodium hyaluronate solution by centrifugation at 12,000 rpm for 20 min at 4° C. Further treatment of high molecular weight hyaluronic acid solution was done with charcoal adsorbed 0.45u filter assembly (Millipore, Millistak +, Activated Carbon minicapsule M40AC23HH3). The flow rate was 14 ml/min. This step removed 60% of the remaining protein.

The flow through from carbon filter was further purified by using continuous mode diafiltration process. Diluted HA solution was pumped (Flow rate 15 ml-20 ml/min) in to cross flow filter holder equipped with 50 kDa cut off polyether sulphone cassette. (Sartocon slice cassette mod. No. 3051465001E-SG). At the beginning of the feeding of the aqueous broth in to filter, the permeate valve was closed in order to recirculate the aqueous broth for several times until the system was stable and no bubbles seen in retentate. The permeate valve was opened after 10 min and the aqueous broth was recirculated in the feed reservoir for additional 10 min to ensure no leakage of HA occurs across the membrane through the permeate valve. After 15 min. permeate and retentate were collected separately. Five equivalent volumes of sterile pyrogen free distilled water were added continuously in the reservoir containing HA solution. This addition of water to HA solution was done at same flow rate as the outflow rate of the filtrate. The inlet pressure of the cross flow filter holder was maintained around 0.5-1.5 bar. The retentate was used for further recovery of hyaluronic acid and permeate was discarded. Essentially, the permeate contained no HA. The retentate was concentrated to original volume and analyzed for purity. The diafiltration step removes 70% of the remaining protein.

The concentrated hyaluronic acid solution: from diafiltration process is finally subjected to 0.22 μm filtration aseptically. (Millipore stericup: SCGPU02RE, PVDF filter material) which renders the product that qualifies for biomedical application. The product thus formed was lyophilized to obtain high molecular wt. medical grade sodium hyaluronate with a residual protein content of 0.031% with respect to HA with a recovery of 51%.

### EXAMPLE 8

The process in Example 1 was scaled up to 10 L. Recovery of HA and purification profile could be reproduced. The final product resulted in a product that had 0.066% protein with respect to HA and the recovery of HA was 82%.

### EXAMPLE 9

### Relative Analysis

The following table indicates the relative analysis:

| | HA purified/ | | HA | | | Total | |
|---|---|---|---|---|---|---|---|
| | treated | Volume | yield | Protein | Total | Protein | % Protein |
| S. No | with | ml | mg/ml | mg/ml | HA mg | mg | w.r.t. HA |
| 1 | IPA | 100 | 3.376 | 0.56 | 337.6 | 56 | 14.22 |
| 2 | Silica gel | 90 | 3.22 | 0.15 | 289.8 | 13.5 | 4.45 |
| 3 | Carbon | 90 | 3.114 | 0.0187 | 280.26 | 1.68 | 0.6 |
| 4 | Dia- | 128 | 1.716 | 0.0014 | 219.6 | 0.179 | 0.075 |
| | filtration | | | | | | |
| | (5×) | | | | | | |
| 5 | 0.22 μm | 128 | 1.96 | 0.0011 | 250.88 | 0.14 | 0.056 |
| | filtration | | | | | | |

“IPA” refers to isopropanol; “S. No” refers to step number.

### EXAMPLE 10

### Product Specification

The final product was tested for its protein, nucleic acid, appearance, pH, glucuronic acid content, molecular weight, IR spectra, chloride content, and moisture content. HA having a molecular weight of 3.0-3.5 million Daltons was produced. The tests mentioned above and the material specifications are as set forth in British Pharmacopoeia 2003. Comparative results are tabulated below:

| SR. NO | TEST | SPECIFICATION | RESULT |
|---|---|---|---|
| 1. | Appearance of | Solution clear; absorbance of | Solution clear; |
| | solution | solution measured at 600 nm is | absorbance of |
| | | not greater than 0.01 | soution measured at |
| | | | 600 nm is 0.004 |
| 2. | IR | The spectrum of the test | Complies |
| | spectrophotometry | substance corresponds to the | |
| | | reference spectrum of sodium | |
| | | hyaluronate | |
| 3. | pH | In the range 5.0-8.5 | 6.65 |
| 4. | Nucleic acids | Absorbance at 260 nm does not | 0.033 |
| | | exceed 0.5 | |
| 5. | Protein | Not more than 0.3%. If | 0.056% |
| | | intended for use in parenteral | |
| | | preparations, not more than | |
| | | 0.1% | |
| 6. | Chlorides | 0.5% (max) | Complies |
| 7. | Loss on Drying | Not more than 20% by weight | 18.2% |
| 8. | Assay | Not less than 95.0% and not | 99.2% |
| | | more than 105.0% of sodium | |
| | | hyaluronate calculated with | |
| | | reference to the dried substance | |

### Molecular Weight Determination

Molecular weight of HA was determined by size exclusion chromatography on Shodex OH Pak SB 804-805HQ column by HPLC. The mobile phase used was 0.1 M NaNO3 at a flow rate of 1 ml/min using a RI detector. The molecular weight was 3.9×10⁶ Da in 24 h

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention.

We claim:

1. A process for producing a hyaluronic acid (HA) or its salt comprising:
(a) providing a bacteria capable of producing HA;
(b) providing a nutrient medium for culturing the bacteria, wherein the nutrient medium comprises a divalent metal ion salt, 1% casein hydrolyzate, and 5% of a carbon source selected from the group consisting of lactose and sucrose;
(c) culturing the bacteria in the nutrient medium; and
(d) fermenting the nutrient medium under aerobic conditions,
wherein the hyaluronic acid has a molecular weight of at least 1 million Da.

2. The process of claim 1, where the fermenting is continuous.

3. The process of claim 1, wherein the divalent metal ions salt is selected from MgSO₄, CuSO₄, MnSO₄, and ZnSO₄.

4. The process of claim 3, wherein the salt is ZnSO₄.

5. The process of claim 1, wherein the fermenting step is conducted for at least 15 hours at a temperature of 30-40° C., an agitation speed of 300-500 rpm, an aeration of 1-3 vvm, and at neutral pH.

6. The process of claim 1, wherein the molecular weight of the HA is at least 3×10⁶ Da after 24 hours of fermenting.

7. The process of claim 1, wherein the HA has a molecular weight in the range of about 3.5×10⁶ Da to 4.0×10⁶ Da, and the process produces a yield of at least 5 g of high molecular weight HA per liter of nutrient medium.

8. The process of claim 1, wherein the bacteria is selected from the group consisting of Streptococcus zooepidemicus, Streptococcus equi and Streptococcus pyogenes.

9. The process of claim 8, wherein the bacteria is Streptococcus zooepidemicus.

10. The process of claim 1, wherein the fermenting step is conducted for at least 15 hours at a temperature of 30-40° C., an agitation speed of 300-500 rpm, an aeration of 1-3 vvm, and at neutral pH.

11. A process for producing a hyaluronic acid (HA) or its salt comprising:
(a) providing a bacteria capable of producing HA;
(b) providing a nutrient medium for culturing the bacteria, wherein the nutrient medium comprises ZnSO₄, casein hydrolyzate, and a sugar selected from the group consisting of lactose and sucrose;
(c) culturing the bacteria in the nutrient medium; and
(d) fermenting the nutrient medium under aerobic conditions,
wherein the hyaluronic acid has a molecular weight of at least 1 million Da;
wherein the process produces a yield of at least 5 g of high molecular weight HA per liter of nutrient medium.

12. The process of claim 11, wherein the nutrient medium comprises 1% casein hydrolyzate and 5% of the sugar.

13. A process for producing hyaluronic acid (HA) or its salt comprising:
(a) providing a hyaluronic acid producing Streptococcus zooepidemicus;
(b) providing a nutrient medium for culturing, wherein the nutrient medium comprises ZnSO₄, 1% casein hydrolyzate, and 5% of a sugar selected from the group consisting of lactose and sucrose;
(c) culturing the Streptococcus zooepidemicus in the nutrient medium; and subsequently
(d) fermenting the medium under aerobic conditions;
wherein the fermenting step is conducted at a temperature of 30-40° C., an agitation speed of 300-500 rpm, an aeration of 1-3 vvm, and at neutral pH.

14. A process for purifying hyaluronic acid (HA) or its salt from a bacterial fermentation broth comprising:
(a) diluting and clarifying the fermentation broth;
(b) precipitating the HA present in the broth with a solvent in amount equal to or less than the volume of the diluted and clarified broth of step (a);
(c) dissolving the precipitated HA or its salt in a solution;
(d) adding silica gel to the HA solution or HA salt solution from step (c) and then removing the silica gel;
(e) treating the HA solution or HA salt solution from step (d) with active carbon; and
(f) diafiltrating the HA solution or HA salt solution from step (e) using a solvent in an amount no more that five times the total volume of the solution from step (e);
wherein the process is conducted in the absence of any detergent or surfactant or formalin, and at neutral pH.

15. The process of claim 14, further comprising converting the HA precipitated in step (b) to its salt, and then homogenizing the HA salt in the solution of step (c).

16. The process according to claim 14, wherein the solvent used in step (b) is selected from isopropanol, ethanol, and acetone.

17. The process according to claim 16, wherein the solvent is isopropanol.

18. The process according to claim 14, wherein the solution in step (c) is 3% sodium acetate.

19. The process according to claim 14, wherein the silica gel is removed by centrifugation in step (d).

20. The process according to claim 14, wherein the active carbon is impregnated on cellulose cartridge in step (e).

21. The process according to claim 14, wherein the solvent in step (f) is sterile pyrogen-free water.

22. The process of claim 14, further comprising:
(g) isolating sterile, purified HA or its salt by filtering through an aseptic filter; and
(h) lyophilizing the HA or its salt until the moisture content is less than 5%.

23. The process according to claim 14, wherein the solvent of step (b) is isopropanol, and the method further comprises (g) lyophilizing the HA or its salt until the moisture content is less than 5%.

24. A process for producing a hyaluronic acid (HA) or its salt comprising:
(a) providing a bacteria capable of producing HA;
(b) providing a nutrient medium for culturing the microbial source, wherein the nutrient medium comprises a divalent metal ion salt, 1% casein hydrolyzate, and 5% of a carbon source selected from the group consisting of lactose and sucrose;
(c) culturing the bacteria in the nutrient medium;
(d) fermenting the nutrient medium under aerobic conditions,
(e) diluting and clarifying the nutrient medium;
(f) precipitating the HA present in the diluted medium with a solvent;
(g) dissolving the precipitated HA or its salt in a solution;
(h) adding silica gel to the HA solution or HA salt solution from step (g) and then removing the silica gel;
(i) treating the HA solution or HA salt solution from step (h) with active carbon; and
(j) diafiltrating the HA solution or HA salt solution from step (i) using a solvent;
wherein the process is conducted in the absence of any detergent or surfactant or formalin, and at neutral pH;
wherein the HA has a molecular weight of at least 1 million Da.

25. A process for producing a hyaluronic acid (HA) or its salt comprising:
(a) providing a bacteria capable of producing HA;
(b) providing a nutrient medium for culturing the bacteria, wherein the nutrient medium comprises zinc, casein hydrolyzate at a concentration of 10 g/L, and a carbon source selected from the group consisting of lactose and sucrose at a concentration of 50 g/L
(c) culturing the bacteria in the nutrient medium; and
(d) fermenting the nutrient medium under aerobic conditions,
wherein the hyaluronic acid has a molecular weight of at least 1 million Da.

26. A hyaluronic acid or its salt produced by the process of claim 1, 7, 13, 14, 24 or 25.
